(19) United States
(12) Patent Application Publication
(10) Pub. No.: US 2017/0245977 A1
(43) Pub. Date: Aug. 31, 2017

(54) TAPERED HELICAL COIL BRONCHIAL VALVE

(71) Applicant: Boston Scientific Scimed, Inc., Maple Grove, MN (US)

(72) Inventors: Daniel J. Foster, Lino Lake, MN (US); Brian L. Schmidt, White Bear Lake, MN (US)

(21) Appl. No.: 15/440,766

(22) Filed: Feb. 23, 2017

Related U.S. Application Data
(60) Provisional application No. 62/300,498, filed on Feb. 26, 2016.

(51) Int. Cl. A61F 2/04; A61B 1/267; A61B 17/12; A61M 27/00; A61F 2/90; A61F 2/07; A61F 2/24; A61B 1/018; A61F 2/88

(52) U.S. Cl. CPC A61F 2/04; A61B 1/018; A61B 1/2676; A61B 17/12104; A61B 17/12145; A61B 17/1215; A61F 2/88; A61F 2/90; A61F 2/07; A61F 2/2427; A61M 27/002; A61F 2002/043; A61B 2017/0034

(57) ABSTRACT

This disclosure concerns systems and methods for tissue volume reduction and control of the flow of substances through the body. Systems according to the various embodiments of the disclosure include check valves formed from wire coils which are deployable through a tubular lumen, such as the working channel of an endoscope, or a catheter.

### CROSS-REFERENCE TO RELATED APPLICATIONS

This application claims the benefit of priority to U.S. Provisional Application Ser. No. 62/300,498, filed Feb. 26, 2016, the disclosure of which is herein incorporated by reference in its entirety.

### FIELD OF THE DISCLOSURE

This application relates to the field of medical devices and medical procedures. More particularly, the application is related to devices and methods for reduction of lung volume.

### BACKGROUND

In certain pulmonary diseases such as emphysema, diseased lung tissue may expand abnormally, compressing the diaphragm and adjacent healthy lung tissue and interfering with normal breathing. With emphysema, effected lung tissue loses its elasticity and ability to effectively expel air during exhalation. The most common treatment for these diseases involves reducing the volume of the affected lung portions, for example by surgically resecting diseased lung tissue. However, surgical lung resection is relatively invasive, time consuming and expensive, and carries several risks including infection, air leakage, excessive bleeding, etc. For this reason, interest has grown in minimally invasive volume reduction procedures, such as endobronchial valves marketed by Pulmonx Corporation (Redwood City, Calif.), and shape-memory coil marketed by PneumRx™ (Mountain View, Calif.). The coil can be deployed into distal bronchi through a bronchoscope and assume a coiled- or birds-nest shape so that, when it is deployed within a length of the distal bronchial tube, the device gathers and forces air from surrounding lung tissue. The shape memory coil, however, is intended to reduce the volume of relatively small portions of lung tissue, and so multiple coils may need to be placed within the lung to achieve the desired reduction in volume.

### SUMMARY OF THE DISCLOSURE

The present disclosure, in its various aspects, provides improved systems and methods for lung volume reduction by providing coiled structures that act as check valves to permit deflation of targeted lung tissue but to prevent reinflation.

In one aspect, the present disclosure relates to a system for regulating the flow of a substance within the body, which includes a wire configured to form a tapered wound coil when unconstrained and configured for insertion into the body of the patient in an elongated unwound configuration. The tapered coil is characterized by a taper of less than one filar diameter (filar diameter being the diameter of the wire, also referred to as the wire gauge) per loop of coil. In other words, a first outer diameter of a first loop is preferably greater than or equal to an inner diameter of an adjacent loop having a second outer diameter larger than the first outer diameter when the coil is unconstrained. By way of example and not limitation, if the wire has a diameter of 1 mm, the taper between adjacent loops of coil (the difference between the outer diameter of a first loop and the inner diameter of an adjacent second loop having a larger outer diameter) is less than 1 mm. The wire may be any suitable diameter, for instance 5, 4, 3, 2, 1, 0.9, 0.8, 0.7, 0.6, 0.5, 0.4, 0.3, 0.25, 0.125, or 0.1 mm; in each case, the taper between adjacent loops of coil will be less than the diameter of the wire. The coil also has a spring constant less than a pressure difference between the inner and outer surfaces of the coil during the flow of substance (for instance, blood, plasma, air, etc.) through a body lumen of the patient in a direction of decreasing taper of the coil (i.e. from the wide end to the narrow end of the coil). By way of example and not limitation, if the substance is air and the coil is disposed in the lung, the coil will open when an alveolar pressure is at least 2 mmHg greater than an atmospheric pressure. This allows the coil to function as a check valve within the patient's body: the normal flow of substance will be sufficient to open the coil and permit the substance to flow through the tapered coil in the direction of the decreasing taper, while the coil limits or prevents the flow of substance in the direction of the increasing taper of the coil. In some cases, the distal end of the wire forms the widest portion of the tapered coil, for instance defining one or more (e.g. a plurality) of adjacent loops of substantially constant diameter. The distal end of the wire also optionally includes one or more of a retentive structure (such as a wing or flap), and/or a ball or rounded tip. In some cases, the proximal end of the wire forms the narrowest portion of the tapered coil and optionally includes a membrane that at least partially obstructs the end and one or more loops of the narrowest portion of the tapered coil. In some cases, the system also includes a device such as a catheter or a bronchoscope, and the wire is insertable through a lumen of the device; the proximal end of the wire, in these embodiments, may be able to reversibly couple to a pushrod disposed in the lumen of the device. The wire can include nitinol, stainless steel, or an alloy or polymer with shape-memory, and/or may have one or more flat surfaces (for instance, the cross section of the wire may be rectangular in some cases). In some embodiments, the coil has a distal end and a proximal end which differs from the distal end in one or more of a spring constant, a wire thickness, and a pitch in the unconstrained configuration. The systems according to this aspect of the disclosure are useful in the prevention of reflux and/or regulation of pulsatile flows in body lumens, and may be used for lung volume reduction.

In another aspect, the present disclosure relates to a method of treating a patient that includes disposing, in a bronchus (which term refers, generally, to passages within the lung, including the bronchi, bronchioles, or other airways) of the patient, a check valve comprising a wire wound into a tapered coil characterized by a taper of less than one filar diameter per loop of coil when unconstrained, a spring constant less than a pressure difference between inner and outer surfaces of the coil occurring during exhalation (i.e. the coil opens when alveolar pressure is at least 2 mmHg above atmospheric pressure). The coil is positioned so that a wide portion is distal to a narrow portion within the bronchus. The coil is deliverable, in some cases, via a bronchoscope or catheter, and may be inserted in an elongated configuration through a working channel of the bronchoscope or lumen of the catheter into the bronchus, where it assumes a programmed tapered coil shape. In some cases, the proximal end of the wire is reversibly coupled to a pushrod within the working channel of the bronchoscope. As described above, the wire can include one or more flat surfaces and, in some cases, may have a rectangular cross-section. The wire can also include a membrane or coating.

In yet another aspect, the present disclosure relates to an apparatus for regulating the flow of a substance within a patient's body comprising a wire configured to form a tapered coil when unconstrained, and which is configured for insertion into the body of the patient in an elongated configuration. The tapered coil is characterized by a taper of less than one filar diameter (filar diameter being the diameter of the wire, also referred to as the wire gauge) per loop of coil. The details of the taper are described in more detail above. The coil also has a spring constant less than a pressure difference between inner and outer surfaces of the coil occurring during exhalation (i.e. the coil opens when alveolar pressure is at least 2 mmHg above atmospheric pressure). The distal end of the wire can form the widest portion of the tapered coil, and may include one or more (e.g. a plurality of) adjacent loops of substantially constant diameter, as described above. Alternatively or additionally, the distal end of the wire can include a retentive structure, such as a flap or wing, which will interact with a wall of a body lumen to help hold the coil in place. As described above, the distal end of the wire also optionally includes a ball or a rounded tip. The proximal end of the wire may define the narrowest portion of the tapered coil, and may include a membrane that at least partially obstructs the end and one or more loops of the narrowest portion of the tapered coil. The wire may be insertable through a lumen of another device, such as a catheter or a bronchoscope, in which case the proximal end of the wire may be reversibly coupled to a pushrod in the lumen of the other device. The wire can also include nitinol. In some cases, the tapering coil has a spring constant, wire diameter or pitch when unconstrained that differs at the proximal and distal ends. As discussed above, the wire can include a flat surface and may be rectangular or square in cross-section.

In yet another aspect, the present disclosure relates to a system including a wire configured to form a tapered coil when unconstrained and a device such as a catheter or an endoscope which has a lumen sized to permit insertion of the wire in a constrained configuration. As described above, the tapered coil is characterized by a taper of less than one filar diameter per loop of coil when unconstrained, and the coil has a spring constant less than a pressure difference between inner and outer surfaces of the coil occurring during the flow of a substance through a body lumen from a wide portion of the coil toward a narrow portion of the coil. This system can be used to reduce reflux in a body lumen as follows: the device is inserted into a body lumen and the wire is disposed so that the wide distal portion of the tapered coil is disposed distally within the lumen relative to the narrow proximal portion of the coil. Once deployed, the coil acts as a check valve, permitting flow through the lumen in the distal-to-proximal direction (i.e. in the direction of reducing taper of the coil) while reducing reflux in the proximal-to-distal direction (i.e. in the direction of increasing taper of the coil). The flow through the lumen is optionally pulsatile or cyclical, and the lumen can be a blood vessel, a heart or chamber thereof, a bronchus, bronchiole or other lung passage, and an esophagus and a shunt, whether naturally occurring or surgically-formed. In each case, the coil is preferably positioned so that taper of the coil decreases in the direction of normal flow (i.e. material flows from the wide end of the coil to the narrow end) and increases in the direction of reflux flow (i.e. material flow is resisted in the opposite direction).

### DRAWINGS

Aspects of the disclosure are described below with reference to the following drawings in which like numerals reference like elements, and wherein:

FIGS. 1 and 2 are schematic depictions of coil valves in closed (1) and open (2) configurations.

FIG. 3 shows a schematic depiction of a coil deployed within a bronchus of a patient.

FIGS. 4A, 46 and 4C are schematic depictions of the deployment of a coil valve through a catheter or bronchoscope.

Unless otherwise provided in the following specification, the drawings are not necessarily to scale, with emphasis being placed on illustration of the principles of the disclosure.

### DETAILED DESCRIPTION

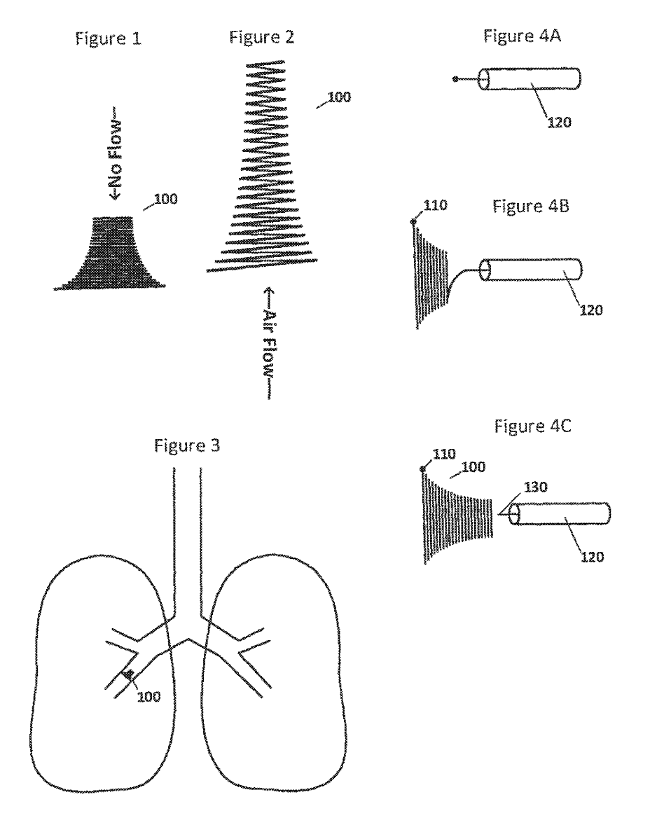

In general, a coil valve 100 according to the present disclosure comprises a single wire wound into an elongated, tapering coil that, when unconstrained (as shown in FIG. 1), has a very small or zero pitch (defined generally as the distance between adjacent windings of the coil or, more rigorously, as the center-to-center distance between adjacent windings of the coil). Coil 100 is characterized by a spring force that is less than a force applied by pressure of the flow against a surface of the coil, e.g., air flow against a surface of the coil 100 during exhalation. Thus, during exhalation, when a pressure difference between the interior and exterior surfaces of the coil 100 exceeds the spring force of the coil 100, the coil 100 elongates and adjacent windings of the coil 100 separate as illustrated in FIG. 2, permitting air to flow between them until the force exerted falls below the restoring force of the coil 100, at which point the coil 100 returns to its closed position. It is generally known that pressure differences as little as 2 mmHg between the alveolar pressure (Pa1v) and atmospheric pressure (Patm) may exist during resting inhalation or exhalation in man. Thus, in preferred embodiments, the coil will open in response to a Pa1v that is at least 2 mmHg greater than Patm, to permit evacuation or exhalation of the portion of the lung downstream of the bronchus in which the coil is deployed.

It should be noted that the elongated, tapering coil designs described herein do not need to be closed at their narrow end to function as check valves. In fact, the coils can be open at both ends, as long as the pressure difference between the interior and exterior surfaces of the coil will exceed the spring constant of the coil during normal use. However, the coils may include, at their narrow end, a covering or other obstructive structure (not shown) that helps limit or prevent the flow of air through the coil in its closed position, thereby increasing the pressure difference between interior and exterior coil surfaces when air is flowing. Alternatively or additionally, airflow through the coil in its closed configuration may be limited by narrowing the narrow end of the coil.

The pitch of the coil 100 in the closed configuration is generally small or zero (i.e. adjacent windings of the wire contact one another along all or most of their length). This is aided, in preferred embodiments, by the use of a shape memory material such as nitinol, and by shape setting the coil 100 to assume a shape with a small or zero pitch. The pitch can be further minimized in some cases by using a wire with one or more flat surfaces (e.g. a wire with a rectangular cross-section, as described in U.S. Pat. No. 9,050,092 by Buiser et al., which is incorporated by reference herein for all purposes).

In use, as shown in FIG. 3, the coil 100 can be deployed in, for instance, a bronchus or other portion of the airway to prevent inflation of a diseased portion of the lung. The coil 100 is deployed so that the wide end is nearest to, and the narrow end is farthest from, the portion of lung being treated; this permits air to flow out of the affected lung portion but limits the rate at which air can flow into the affected lung region (or, if the coil includes a covering or tapers to a narrow point, prevents air from returning to the affected lung region). The coil 100 can, advantageously, be delivered to the lung by means of a catheter or bronchoscope 120, as illustrated in FIGS. 4A, 46 and 4C. The distal end of the coil 100, which forms the base of the valve, is preferably configured to form a loop (or a plurality of loops) having a diameter greater than the inner diameter of the bronchus into which the coil 100 is deployed, so it exerts a radially-outward retentive force on the bronchus. Alternatively, the distal end of the coil 100 is coupled to an expandable scaffold or stent (not shown). The distal end of the coil 100 also optionally includes a ball tip 110 to prevent trauma to the bronchus during deployment. The coil 100 can be advanced through the catheter or bronchoscope 120 by a pushrod, which can be joined to the proximal end of the coil 100 by a severable joint 130, which can be mechanically severable or electrolytically severable. The coil 100 can also include, at its distal end, one or more retention-aiding features, such as wings, etc. (not shown).

While not shown, it should also be noted that the mechanical properties of the coil may vary across its length, for example the spring constant may be relatively lower near the narrow end of the coil, and, similarly, the thickness of the wire may be relatively greater at the widest end of the coil, to facilitate retention.

Although the foregoing examples have focused on lung volume reduction, the systems and methods of the present disclosure can be adapted to control fluid flows in other body lumens. For example, the valves of the present disclosure may be particularly useful in limiting retrograde flows or reflux in settings where flows are pulsatile or cyclical, such as heart valves, blood vessels, etc.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present disclosure are described above. It is, however, expressly noted that the present disclosure is not limited to those embodiments, but rather the intention is that additions and modifications to what was expressly described herein are also included within the scope of the disclosure. Moreover, it is to be understood that the features of the various embodiments described herein were not mutually exclusive and can exist in various combinations and permutations, even if such combinations or permutations were not made express herein, without departing from the spirit and scope of the disclosure. In fact, variations, modifications, and other implementations of what was described herein will occur to those of ordinary skill in the art without departing from the spirit and the scope of the disclosure. As such, the disclosure is not to be defined only by the preceding illustrative description.

1. A method of treating a patient, comprising:
disposing, in a bronchus of a patient, a check valve comprising a wire wound into a tapered coil,
wherein (a) the tapered coil is characterized by a taper of less than one filar diameter per loop of coil when unconstrained, (b) the coil has a spring constant less than a pressure difference between inner and outer surfaces of the coil occurring during exhalation, and (c) the coil is positioned so that a wide portion of the coil is distal to a narrow portion of the coil within the bronchus.

2. The method of claim 1, wherein the step of disposing the check valve in the bronchus of the patient includes inserting the wire through a bronchoscope.

3. The method of claim 2, wherein the wire is in an elongated configuration in a working channel of the bronchoscope, and assumes a tapered coiled shape when advanced out of the channel into the bronchus.

4. The method of claim 3, wherein a proximal end of the wire is reversibly coupled to a pushrod disposed within the working channel of the bronchoscope.

5. The method of claim 1, wherein the wire includes a membrane or a coating.

6. An apparatus for regulating a flow of a substance within a body of a patient, comprising:
a wire configured to form a tapered coil when unconstrained, the wire being further configured for insertion into the body of the patient in an elongated configuration,
wherein (a) the tapered coil is characterized by a taper of less than one filar diameter per loop of coil when unconstrained, and (b) the coil has a spring constant less than a pressure difference between inner and outer surfaces of the coil occurring during exhalation.

7. The apparatus according to claim 6, wherein a distal end of the wire forms the widest portion of the tapered coil.

8. The apparatus according to claim 7, wherein the distal end of the wire includes a plurality of the loops of the coil of substantially constant diameter.

9. The apparatus according to claim 6, wherein a distal end of the wire includes a retentive structure.

10. The apparatus according to claim 6, wherein the distal end of the wire includes a ball or a rounded tip.

11. The apparatus according to claim 6, wherein a proximal end of the wire forms the narrowest portion of the tapered coil.

12. The apparatus according to claim 11, wherein the proximal end of the wire includes a membrane that at least partially obstructs the narrowest portion of the tapered coil.

13. The apparatus according to claim 6, wherein the wire is insertable through a lumen of a device selected from the group consisting of a catheter and bronchoscope, and a proximal end of the wire is configured to reversibly couple to a pushrod disposed in the lumen of the device.

14. The apparatus according to claim 6, wherein the wire includes nitinol.

15. The apparatus according to claim 6, wherein the coil has a distal end and a proximal end which differs from the distal end in one or more of a spring constant, a wire thickness, and a pitch in the unconstrained configuration.

16. The system according to claim 6, wherein the wire has a rectangular cross-section.

17. A system, comprising:
a wire configured to form a tapered coil when unconstrained; and
a device selected from the group consisting of a catheter and an endoscope, the device having a lumen sized to permit insertion of the wire in an elongated configuration,
wherein (a) the tapered coil is characterized by a taper of less than one filar diameter per loop of coil when unconstrained, and (b) the coil has a spring constant less than a pressure difference between inner and outer surfaces of the coil occurring during the flow of a substance through a body lumen from a wide portion of the coil toward a narrow portion of the coil.

18. A method of reducing reflux in a body lumen using the system of claim 17, comprising the steps of:
inserting the device into a body lumen; and
disposing, through the device, the wire, such that a wide distal portion of the tapered coil is disposed distally within the lumen relative to a narrow proximal portion of the tapered coil, thereby reducing reflux in the proximal-to-distal direction in the lumen.

19. The method according to claim 18, wherein the flow of the substance through the body lumen is pulsatile or cyclical in nature.

20. The method according to claim 18, wherein the body lumen is selected from the group consisting of a blood vessel, a heart, a bronchus, a bronchiole, an esophagus and a shunt.
